(19) United States
(12) Patent Application Publication
(10) Pub. No.: US 2010/0256461 A1
(43) Pub. Date: Oct. 7, 2010

(54) APPARATUS AND METHODS FOR EVALUATING PHYSIOLOGICAL CONDITIONS OF TISSUE

(75) Inventors: Zeid Mohamedali, Qualicum Beach (CA); Luya LI, Coquitlam (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: URODYNAMIX TECHNOLOGIES LTD., Vancouver, BC (CA)

(21) Appl. No.: 12/598,420

(22) PCT Filed: May 1, 2008
(86) PCT No.: PCT/CA08/00821

§ 371 Date: Oct. 30, 2009

Related U.S. Application Data
(60) Provisional application No. 60/915,399, filed on May 1, 2007.
(60) Provisional application No. 61/033,737, filed on Mar. 4, 2008.

(51) Int. Cl. A61B 5/00; A61B 8/00

(52) U.S. Cl. 600/301; 600/437

(57) ABSTRACT

Sensors for use in evaluating physiological conditions of tissue are provided on a structure having an opening for receiving a probe, finger or the like. The sensors include near infrared spectroscopy sensors in some embodiments. The structure may comprise a flexible sleeve that may be worn on the finger of a physician performing a rectal exam of the prostate, or placed over a rectal ultrasound probe for example. In an embodiment, a controller processes data from sensors to provide information relevant to blood flow in different parts of a subject's prostate gland.

### REFERENCE TO RELATED APPLICATIONS

This application claims priority from U.S. patent application No. 61/033,737 filed 4 Mar. 2008 entitled DIGITALLY-SUPPORTED PHYSIOLOGICAL SENSORS and U.S. patent application No. 60/915,399 filed 1 May 2007 entitled DIGITALLY-SUPPORTED PHYSIOLOGICAL SENSORS. For purposes of the United States of America, this application claims the benefit under 35 U.S.C. §119 of U.S. patent application No. 61/033,737 filed 4 Mar. 2008 entitled DIGITALLY-SUPPORTED PHYSIOLOGICAL SENSORS and U.S. patent application No. 60/915,399 filed 1 May 2007 entitled DIGITALLY-SUPPORTED PHYSIOLOGICAL SENSORS, both of which are hereby incorporated herein by reference.

### TECHNICAL FIELD

The invention relates to medical devices and, in particular to devices for measuring physiological information. Particular embodiments of the invention provide devices for assessing conditions of the prostate gland and/or associated tissues.

### BACKGROUND

Prostate cancer and other prostate pathologies affect a large proportion of men. Current techniques for evaluating the condition of the prostate include digital rectal examination and blood tests for prostate specific antigen (PSA). The efficacy of digital rectal examination depends on the skill and sensitivity of the physician performing the procedure.

Near Infrared Spectroscopy (“NIRS”) is a technique which involves emitting near infrared (“NIR”) light and receiving the NIR light after it has passed through a tissue or other medium of interest. NIRS can be applied to study and monitor biochemical compounds in the body. Emitted NIR light penetrates skin and other tissues and some of it is absorbed by biochemical compounds which have an absorption spectrum in the NIR region. NIR light which is not absorbed is scattered. Each biochemical compound has a different absorption spectrum. It is possible to estimate the concentration of biochemical compounds in the tissues by measuring characteristics of NIR light that has been detected after it has passed through the tissues.

There is a need for practical and cost-effective systems that provide physicians and other medical practitioners with additional information regarding conditions that may affect the health of their patients. There is a need for practical and cost-effective systems capable of providing physicians with more information regarding the prostate and associated tissues.

### SUMMARY OF THE INVENTION

Aspects of the invention and features of specific embodiments of the invention are described below

According to one aspect of the invention, an apparatus for evaluating one or more physiological conditions of a subject's tissue is provided. The apparatus has one or more sensors. Support means support the one or more sensors removably in place over a member insertable in a body cavity of the subject. A control unit is operatively connected to the one or more sensors. When a user places the support means supporting the one or more sensors over the member in the body cavity in proximity to the subject's tissue, the one or more sensors can sense and transmit to the control unit information relevant to the physiological condition of the subject's tissue.

According to another aspect of the invention, an apparatus for evaluating one or more physiological conditions of a subject's prostate and/or associated tissue is provided. The apparatus has a sleeve dimensioned to be worn on a finger of a user. The sleeve comprising a proximal end open to receive the user's finger. One or more sensors are supported by the sleeve. A control unit is operatively connected to the one or more sensors. When the user places the one or more sensors supported by the sleeve on the user's finger in proximity to the subject's prostate, the one or more sensors can sense and transmit to the control unit information relevant to the one or more physiological conditions of the subject's prostate and/or associated tissue.

According to a further aspect of the invention, an apparatus for evaluating one or more physiological conditions of a subject's prostate and/or associated tissue is provided. The apparatus has a sleeve dimensioned to be fitted over an anal ultrasound probe. The sleeve has a proximal end open to receive the anal ultrasound probe. One or more sensors are supported by the sleeve. A control unit is operatively connected to the one or more sensors. When a user places the one or more sensors supported by the sleeve fitted over the anal ultrasound probe in proximity to the subject's prostate, the one or more sensors can sense and transmit to the control unit information relevant to the one or more physiological conditions of the subject's prostate and/or associated tissue.

According to yet another aspect of the invention, an apparatus for evaluating one or more physiological conditions of a subject's prostate and/or associated tissue. The apparatus comprises an anal probe which supports a plurality of ultrasonic transducers and a plurality of near infrared (NIR) light sensors. An ultrasound control unit is operatively connected to the ultrasonic transducers, and an NIR light sensor control unit is operatively connected to the NIR light sensors. When a user places the probe in proximity to the subject's prostate, the ultrasonic transducers can obtain and transmit to the ultrasound control unit information for generating an ultrasound image of the subject's prostate and/or associated tissue, and the NIR light sensors can sense and transmit to the NIR light sensor control unit information relevant to the one or more physiological conditions of the subject's prostate and/or associated tissue corresponding to the ultrasound image.

According to another aspect of the invention, a method for evaluating one or more physiological conditions of a subject's tissue is provided. The method includes the steps of:
(a) providing a support means for supporting one or more sensors removably in place over a member insertable in a body cavity of the subject;
(b) fitting the support means supporting the one or more sensors over the member;
(c) inserting the member into the subject's body cavity;
(d) positioning the one or more sensors in proximity to the subject's tissue; and
(e) detecting with the one or more sensors information relevant to the physiological condition of the subject's tissue.

According to a further aspect of the invention, a method for evaluating one or more physiological conditions of a subject's prostate and/or associated tissue is provided. The method includes the steps of:
(a) providing a sleeve supporting one or more sensors;
(b) wearing the sleeve on the user's finger;
(c) inserting the user's finger into the subject's anal cavity;
(d) positioning the one or more sensors in proximity to the subject's prostate; and
(e) detecting with the one or more sensors information relevant to the one or more physiological conditions of the subject's prostate and/or associated tissue.

According to yet another aspect of the invention, a method for evaluating one or more physiological conditions of a subject's prostate and/or associated tissue is provided. The method includes the steps of:
(a) providing a sleeve supporting one or more sensors;
(b) fitting the sleeve over an anal ultrasound probe having ultrasonic transducers;
(c) inserting the anal ultrasound probe into the subject's anal cavity;
(d) positioning the one or more sensors and the ultrasonic transducers in proximity to the subject's prostate;
(e) detecting with the ultrasonic transducers information for generating an ultrasound image of the subject's prostate and/or associated tissue; and
(f) detecting with the one or more sensors information relevant to the one or more physiological conditions of the subject's prostate and/or associated tissue corresponding to the ultrasound image.

According to another aspect of the invention, a method for evaluating one or more physiological conditions of a subject's prostate and/or associated tissue is provided. The method includes the steps of:
(a) providing an anal probe supporting a plurality of ultrasonic transducers and a plurality of near infrared (NIR) light sensors;
(b) inserting the anal probe into the subject's anal cavity;
(c) positioning the ultrasonic transducers and the NIR light sensors in proximity to the subject's prostate;
(d) detecting with the ultrasonic transducers information for generating an ultrasound image of the subject's prostate and/or associated tissue; and
(e) detecting with the NIR light sensors information relevant to the one or more physiological conditions of the subject's prostate and/or associated tissue corresponding to the ultrasound image.

### BRIEF DESCRIPTION OF THE DRAWINGS

The accompanying drawings illustrate non-limiting embodiments of the invention.

FIG. 1 is a schematic diagram of apparatus according to an example embodiment of the invention.

FIG. 2 is a diagram schematically showing a finger-mounted sensor assembly of the apparatus of FIG. 1 being worn on a finger.

FIG. 3 shows the apparatus of FIG. 1 being deployed to obtain measurements of conditions in a subject's prostate gland.

FIG. 3A shows apparatus according to an embodiment of the invention.

FIG. 4 is a schematic diagram illustrating an embodiment of the invention that performs near infrared spectroscopy.

FIGS. 5A through 5C are possible arrangements for sensors and light sources in an array of sensors and light sources.

FIG. 6 is a schematic diagram of apparatus according to an alternative embodiment of the invention.

FIG. 7 is a schematic diagram of apparatus according to a further alternative embodiment that comprises an ultrasound imaging device having an anal probe.

FIG. 8 is a schematic illustration showing a combined ultrasound and NIRS anal probe.

FIG. 9 is a sketch illustrating a general manner in which a depth of a location to which a light-source-sensor pair is most sensitive varies with separation of the light-source-sensor pair.

FIG. 10 illustrates schematically a display that may be produced according to an example embodiment that combines ultrasound and NIRS.

FIGS. 11A, 11B, 11C and 11D illustrate variations in a parameter derived from NIRS data for different sections of a subject's prostate.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

This invention provides apparatus for evaluating physiological conditions of individuals. The apparatus comprises one or more sensors that can be removably supported in place by support means over a member insertable in a body cavity of the subject. The support means may be a flexible sleeve. The insertable member may be a probe or a physician's finger. The sensors can then be placed in proximity to tissues of a subject to permit the sensors to detect one or more physiological characteristics of the tissues. Some embodiments provide an array of sensors capable of measuring information about the tissues of the subject that can be processed to generate an image of the tissues or an image of the variation of some characteristic of the tissues with position.

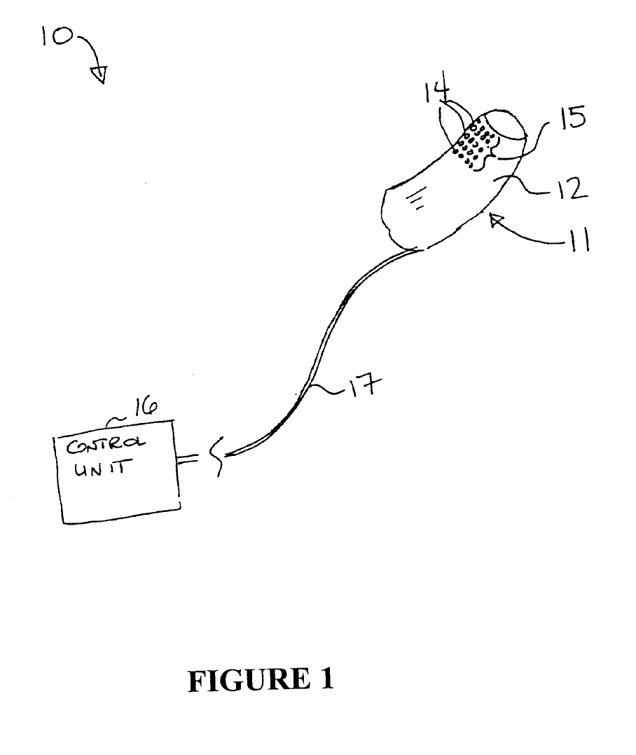

FIG. 1 is a schematic illustration of apparatus 10 according to one embodiment of the invention. Apparatus 10 comprises a finger-mounted sensor assembly 11. In the illustrated embodiment, assembly 11 comprises a sleeve 12 carrying one or more sensors 14. In the illustrated embodiment, sensors 14 are arranged in a two-dimensional array 15. Signals from sensors 14 are carried to a control unit 16 by one or more cables 17. Cables 17 may also carry power, control signals, radiation, or other requirements of sensors 14 from control unit 16 to sensor array 15.

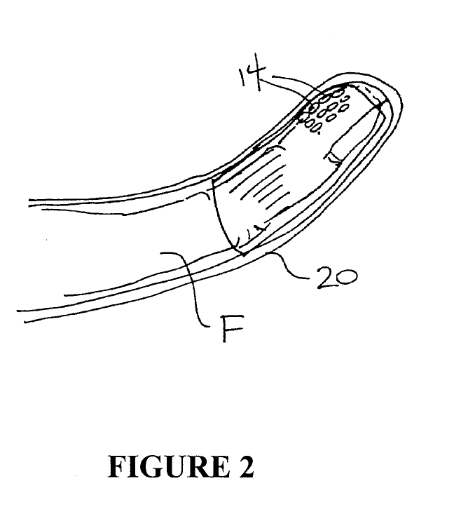

Sleeve 12 is dimensioned to be worn on a physician's finger. Sleeve 12 may comprise an elastic tube which fits smoothly around a physician's finger and supports sensor array 15. One end of sleeve 12 is open to receive the physician's finger. The other end of sleeve 12 may be closed, like the finger of a glove, or open. FIG. 2, shows apparatus 10 with sleeve 12 on a physician's finger F. FIG. 2 also shows a disposable sheath 20, which may be the finger of a glove, for example, worn over sleeve 12. The material of sheath 20 is selected to not interfere excessively with the operation of sensors 14. For example, where sensors 14 detect radiation of some kind, the material of sheath 20 may be selected to be transparent, or nearly transparent to the radiation.

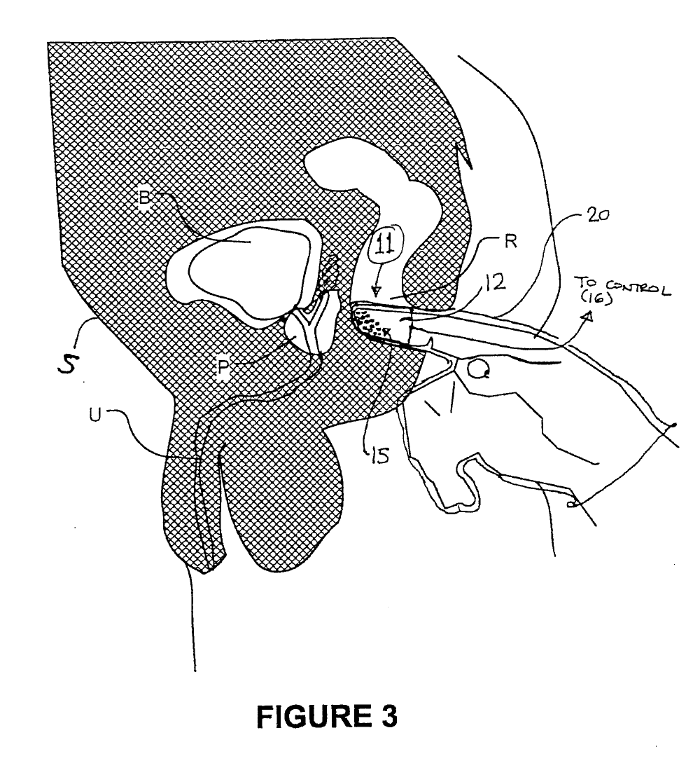

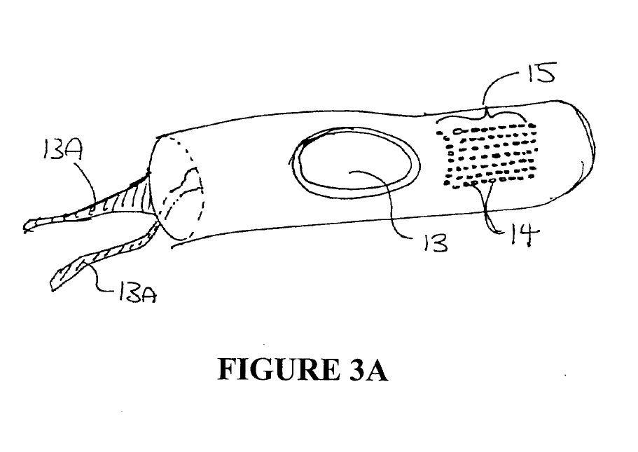

FIG. 3 shows schematically apparatus 10 being used to obtain information regarding a subject's prostate gland P. Finger F is inserted into the rectum R of a subject S. Array 15 extends over the ventral surface of at least the tip of finger F. Prostate P is adjacent to array 15 through the wall of the subject's colon. In this configuration, array 15 can sense and transmit to control unit 16 information relevant to the physiological condition of prostate P and/or associated tissues. The nature of the information delivered to control unit 16 will depend upon the types of sensor 14 provided on sleeve 12. Sensors 14 may be of one or more different types.

Sensors 14 may include one or more of the following types of sensor, for example:
sensors that receive near infrared light emitted by a source of infrared light (the source may be on sleeve 12 or a separate external source may be provided);
light sensors;
ultrasound sensors;
temperature sensors; and
force (pressure) sensors.

Sensors 14 may be arranged densely enough to provide data that can be processed to provide “images” of the properties that they detect. For example, sensors 14 may be arranged to have a spacing between adjacent sensors 14 of about 1½ mm or less. In some embodiments, sensors 14 have a spacing between nearest-neighbors of about ¾ mm or less, or ½ mm or less.

In some embodiments, sensors 14 include sensors that detect signals indicative of physiological conditions at a plurality of different distances from sensors 14. Such embodiments may permit the acquisition of data from which three-dimensional images may be generated (or for which parameters somehow indicative of one or more physiological conditions can be measured at points of a three-dimensional point-cloud).

Preferably, sleeve 12 and array 15 are flexible and adapt to the contours of Finger F such that the physician retains some “feel” for the surrounding tissues even while wearing sleeve 12. In some embodiments, as shown for example in FIG. 3A, sleeve 12 comprises an aperture 13. Sleeve 12 can first be pulled onto a physician's finger so that the sensitive part of the physician's fingertip can feel tissues of interest through aperture 13. When the physician's finger is in the desired position with respect to the tissues of interest, the physician can pull sleeve 12 farther onto his or her finger so that sensor array 15 is held against the tissues of interest by the physician's finger. One or more cords 13A or other elongated members may extend from sleeve 12 to the vicinity of the physician's wrists to permit sleeve 12 to be pulled further onto the physician's finger while the finger is inserted into the subject's rectum or otherwise engaging tissues of interest.

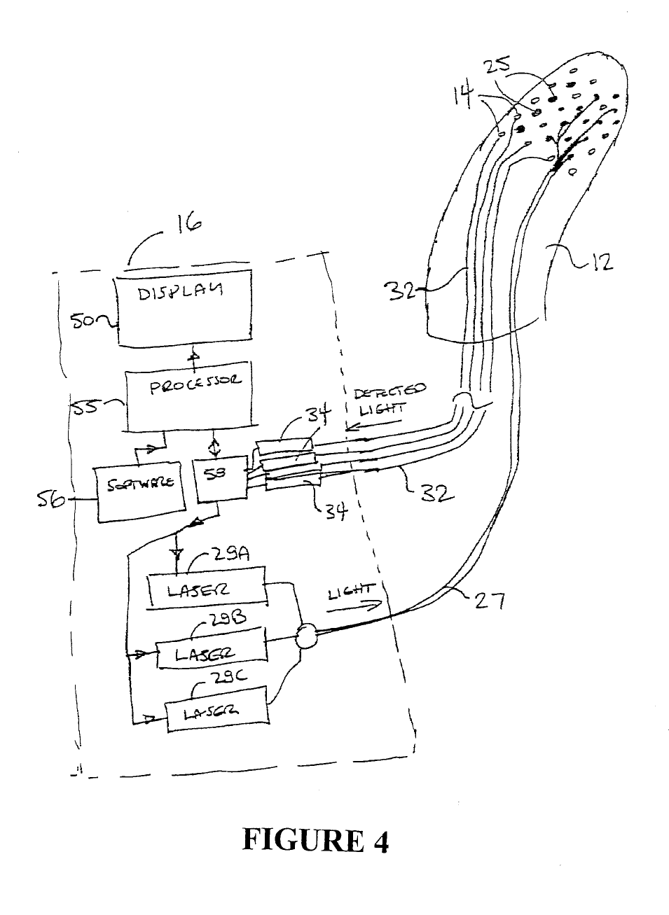

FIG. 4 shows a block diagram of an example embodiment in which sensors 14 comprise sensors of a type useful for performing near-infrared spectroscopy (NIRS) on tissues of a subject. Each sensor 14 comprises an infrared light detector that receives infrared radiation from a light source. In the illustrated embodiment, a plurality of light sources 25 are disposed on sleeve 12.

Light sources 25 emit radiation of two or more wavelengths (typically NIRS uses light in two or three narrow wavelength bands). In the illustrated embodiment, light sources comprise optical fibers 27 that carry light from laser diodes or other suitable sources of light in controller 16. In the illustrated embodiment there are three laser diodes 29A, 29B and 29C. The multiple wavelengths may be combined and carried in a single set of optical fibers 27, as illustrated or separate sets of optical fibers may be provided for different wavelengths.

In the illustrated embodiment, sensors 14 comprise optical fibers 32 that receive light and carry the light to photo diodes, phototransistors, or other light detectors 34 that generate electrical signals indicative of the intensity of light incident at the distal ends of optical fibers 32. In this embodiment, sensor assembly 11 does not need to include any active electronic devices.

In other embodiments, sensors 14 comprise active electronic devices mounted on or to sleeve 12 that generate signals indicative of the intensity of light incident upon them.

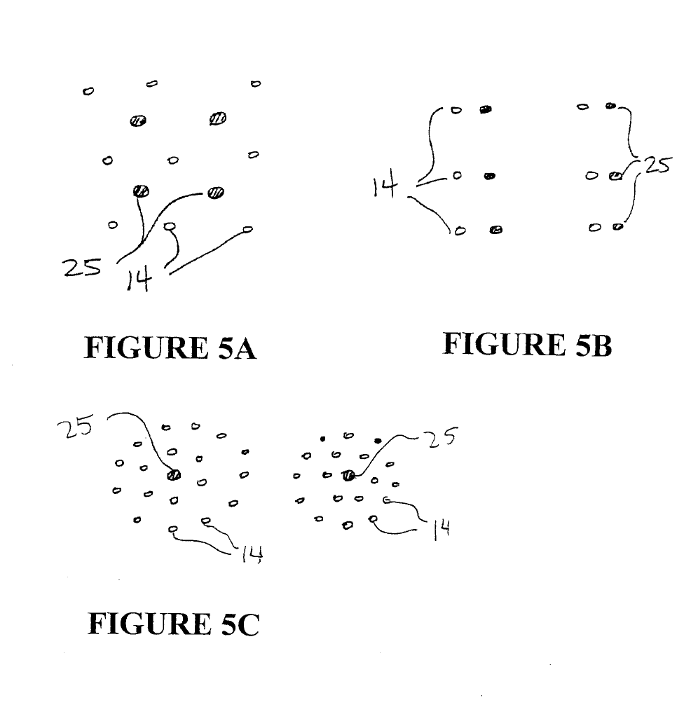

In some embodiments, each sensor 14 is paired with a light source 25. In some embodiments, a light source 25 is associated with a plurality of sensors 14. In some such embodiments, the plurality of sensors 14 are spaced apart from the corresponding light source by a plurality of different distances. FIGS. 5A, 5B and 5C show a number of possible arrangements of light sources 25 and sensors 14 (comprising light detectors).

In some embodiments, there are two or more sets of light sources 25 that are independently controllable or differently modulated (so that light from different sets of light sources 25 can be distinguished). In such embodiments, measurements at various depths in the subject's tissues may be achieved by detecting at a sensor 14 light originating from different light sources 25 at different distances from the sensor 14.

It is desirable to prevent sensors 14 from picking up signals relating to tissues in finger F. Where sensors 14 sense light, this may be accomplished, for example, by one or more of:
Making the portions of sleeve 12 underlying array 15 opaque;
Providing an opaque layer between sensors 14 and finger F;
Making sensors 14 have a directional characteristic such that they are not sensitive to radiation incident from the direction of finger F; and,
Making light sources 25 directional such that the tissues of Finger F are not illuminated.

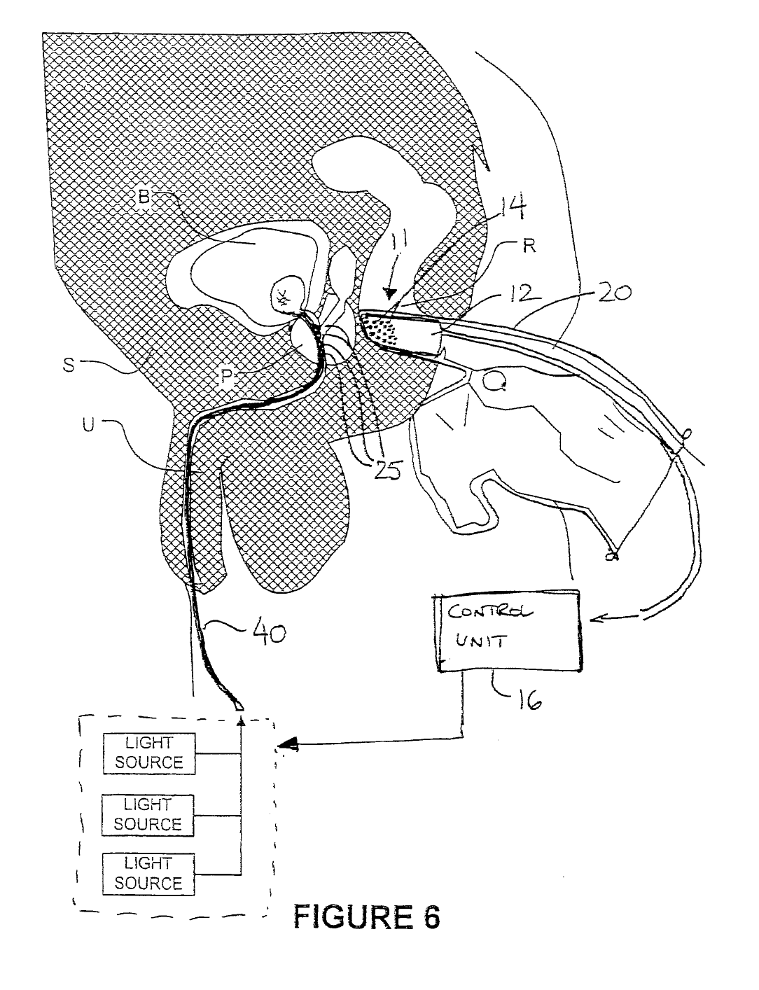

It is not mandatory that light sources 25 be on assembly 11. Instead, light sources 25 may be supported on an element independent of assembly 11. FIG. 6 illustrates an alternative embodiment wherein one or more light sources 25 is provided on a catheter 40 inserted through the subject's urethra U into prostate P. Light from light source 25 passes through tissues of prostate P and is detected by sensors 14 of assembly 11. In the illustrated embodiment, there are a plurality of independently-controllable light sources 25 on catheter 40.

Control unit 16 may comprise a display 50 on which operational information relating to system 10 and/or data (including images in some embodiments) acquired by system 10 may be displayed. A data store 52 permits storage of data acquired from a subject (and/or information derived from that data). In the illustrated embodiment, control unit 16 comprises a microprocessor 55 executing stored instructions 56 that cause microprocessor 55 to control the operation of apparatus 10 by way of suitable interfaces 58.

Where sensors 14 are applied to detect light from suitable light sources to perform NIRS, the signals output by sensors 14 may be processed to yield values indicative of the concentrations of bio-compounds such as:

Total Haemoglobin (HbTot);

Oxygenated Haemoglobin (HbO₂);

Non-oxygenated haemoglobin (Hb);

Cytochrome C Oxydase (Cyt);

Myoglobin (Mb); and,

other chromophores;

at points within the subject's tissues. In some embodiments, the signals output by sensors 14 may be processed to detect the presence of and/or measure concentrations of exogenous markers or other chemical agents that may be introduced into the tissues of subject S for the purpose of imaging or the detection of certain conditions. In some embodiments the signals output by sensors 14 are processed to provide indications of blood flow or blood volume at locations within the subject's tissues. Such measures can be useful in the diagnosis of conditions which involve increased vascularization or increased metabolic activity in the vicinity of tumors or other lesions.

Apparatus and methods as described herein may be applied to detect conditions such as cancers, tumors, increased vascularity or the like and/or to differentiate tissues affected by such conditions from normal tissues and/or to locate tumors, areas of increased vascularity or the like.

Transrectal ultrasound imaging may also be applied to study the prostate. An example system for transrectal ultrasound imaging is described in Fenster, A. et al., Three-dimensional ultrasound imaging systems for prostate cancer diagnosis and treatment Instrumentation & Measurement Magazine, IEEE Vol. 1, No. 4, December 1998, pp. 32-35. An assembly 11 as described above may be carried on an anal ultrasound probe to provide simultaneous ultrasound imaging of the prostate and surrounding tissues and NIRS studies of the prostate.

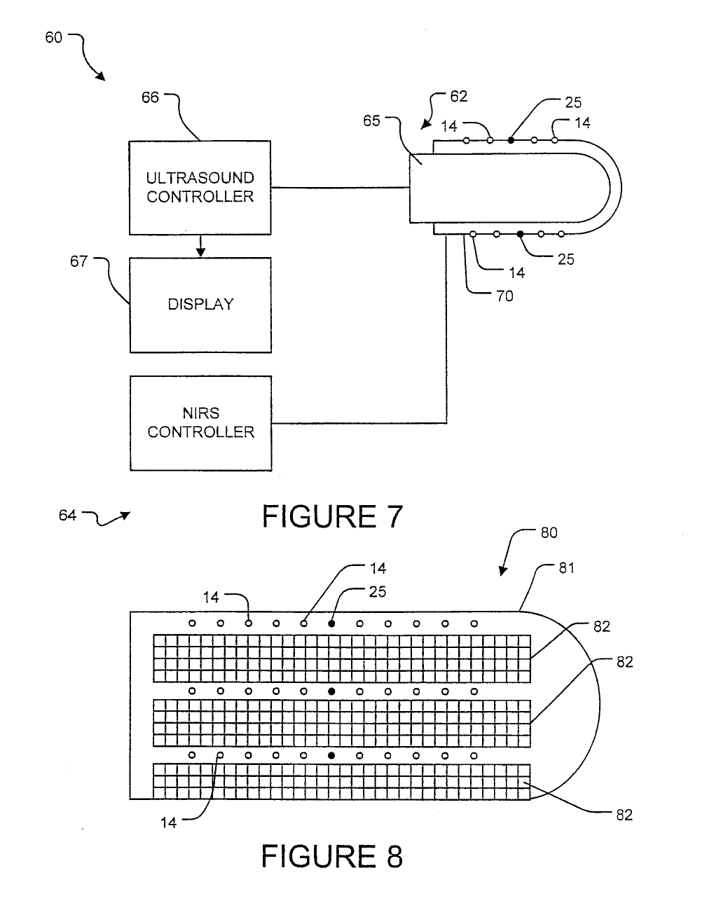

FIG. 7 shows apparatus 60 comprising an ultrasound system 62 and a NIRS system 64. Ultrasound system 62 comprises an anal probe 65 connected to an ultrasound controller 66 which includes a display 67. Controller 66 provides the necessary circuits to drive ultrasound transducers (not shown) of anal ultrasound probe 65 and to process acoustic signals reflected from structures with a subject's body to provide an ultrasound image on display 67. Such ultrasound systems are commercially available.

NIRS system 64 comprises a sleeve 70 which supports light sources 25 and sensors 14 as described above. Sleeve 70 slips over anal probe 65. Sleeve 70 is substantially acoustically transparent so that it does not interfere with ultrasound imaging. Water or a suitable acoustic gel 71 may be provided between sleeve 70 and anal probe 65 to facilitate clean transmission of acoustic signals between anal probe 65 and the subject's tissues. Sleeve 70 may comprise a flexible material or may be rigid or semi-rigid.

A controller 16 as described above drives light sources 25 to emit light of wavelengths suitable for detecting one or more biochemical compounds of interest by NIRS and processes signals received from sensors 14.

Sleeve 70 may be constructed to permit it to be aligned in a known way with anal ultrasound probe 65. For example:
sleeve 70 may bear indicia such as a mark 73 that can be aligned with a feature or mark on anal probe 65 such that sensors 14 have known locations relative to ultrasonic transducers of anal ultrasound probe 65.
sleeve 70 and anal ultrasound probe 65 may be shaped such that they can fit together only in one (or only in selected) relative orientations.
   Where sensors 14 have known locations relative to ultrasonic transducers of anal ultrasound probe 65 it is possible to correlate the locations for which concentrations of bio-compounds are determined from the outputs of sensors 14 to locations shown in ultrasound images obtained using anal ultrasound probe 65.

Synergies may be obtained by providing an ultrasound probe which includes sensors 14 or both sensors 14 and light sources 25 for use in NIRS. FIG. 8 shows schematically a combined ultrasound and NIRS anal probe 80. Probe 80 has a body 81 that supports an array of ultrasound transducers 82. Body 81 also supports a number of NIRS light sources 25 and a number of sensors 14. In the illustrated embodiment, light sources 25 and sensors 14 are arranged in rows between blocks of ultrasound transducers 82. In alternative embodiments:
light sources 25 and sensors 14 are interspersed among ultrasound transducers 82.
light sources 25 and sensors 14 are acoustically transparent and overlie ultrasound transducers 82.
ultrasound transducers 82 are substantially transparent at the wavelengths detected by sensors 14 and overlie sensors 14 and/or light sources 25.
light sources 25 and sensors 14 are arranged in bands that partially or entirely encircle anal probe 80 (which may have a round or rounded cross-sectional shape).
light sources 25 and sensors 14 are arranged in bands that spiral around anal probe 80.

A pair made up of a NIRS light source and detector is most sensitive to the presence of bio-compounds at a location in the subject's tissues that is between the light source and detector and is at a depth depending on the spacing between the light source and detector. The size, shape and precise location of the location at which a light-source-sensor pair is most sensitive is determined by a range of factors including the light emission pattern of the light source 25 and any variations in sensitivity with direction for sensor 14.

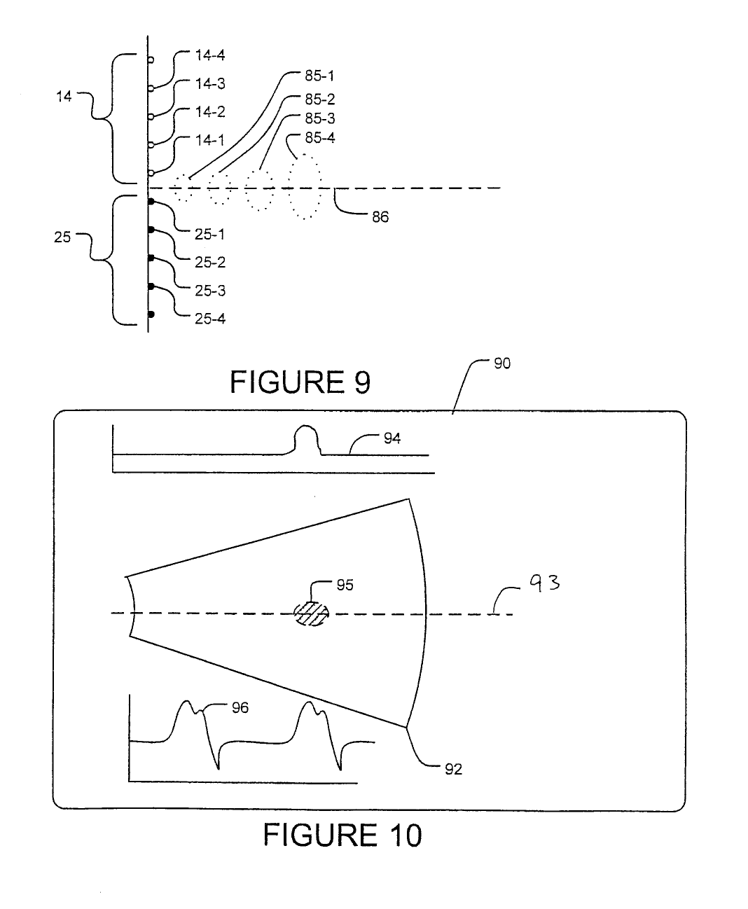

FIG. 9 shows schematically a number of pairs of light sources 25 and sensors 14 and the location at which each pair is most sensitive. In FIG. 9:
the pair of light source 25-1 and sensor 14-1 is most sensitive at a location 85-1;
the pair of light source 25-2 and sensor 14-2 is most sensitive at a location 85-2;
the pair of light source 25-3 and sensor 14-3 is most sensitive at a location 85-3;
and so on.
   Locations 85-1, 85-2, 85-3 are collectively locations 85.

FIG. 9 also shows an image plane 86 for an ultrasound image in one imaging mode. It can be seen that each of locations 85 corresponds to a location in image plane 86. Some embodiments include a display that displays an ultrasound image and is also configured to mark on the ultrasound image locations 85 or a line along which locations 85 are situated. For example, FIG. 10 shows a display 90 displaying an ultrasound image 92. A line 93 is displayed on display 90 and indicates the direction of locations 85 for one or more light-source-sensor pairs. A graph 94 indicates the variation in a parameter calculated by control unit 16 from outputs of sensors 14 as a function of the position along line 93. The parameter may, for example, comprise a concentration of a bio-compound. In the illustrated embodiment, the parameter comprises a measure of total haemoglobin (HbTot). It can be seen from graph 94 that HbTot is increased in an area corresponding to an anomaly 95 visible in ultrasound image 92.

In some embodiments display 90 includes elements of a graphical user interface GUI. In such embodiments, a user may be able to identify a location of interest in ultrasound image 92, for example by positioning a cursor using a pointing device, touching on a touch-sensitive part of display 90 or the like. In response to identification of a location of interest, controller 16 may identify one or more light-source-sensor pairs that yield information relevant to the identified location. Control unit 16 then computes one or more parameter values (such as estimated concentrations of one or more bio-compounds) from outputs corresponding to the identified light-source-sensor pairs. The parameter values corresponding to the selected location may be displayed on display 90. In the illustrated embodiment, a graph 96 illustrates variation in a parameter (for example, [HbTot]) with time for the currently-selected location.

As shown in FIG. 8, a combined ultrasound and NIRS anal probe may have pairs of light sources 25 and sensors 14 facing in different directions. Such light-source-sensor pairs may be oriented to detect bio-compounds or other materials in different sections of the prostate. In some embodiments, a combined ultrasound and NIRS anal probe has sufficient light-source-sensor pairs to perform NIRS on quadrants of the prostate.

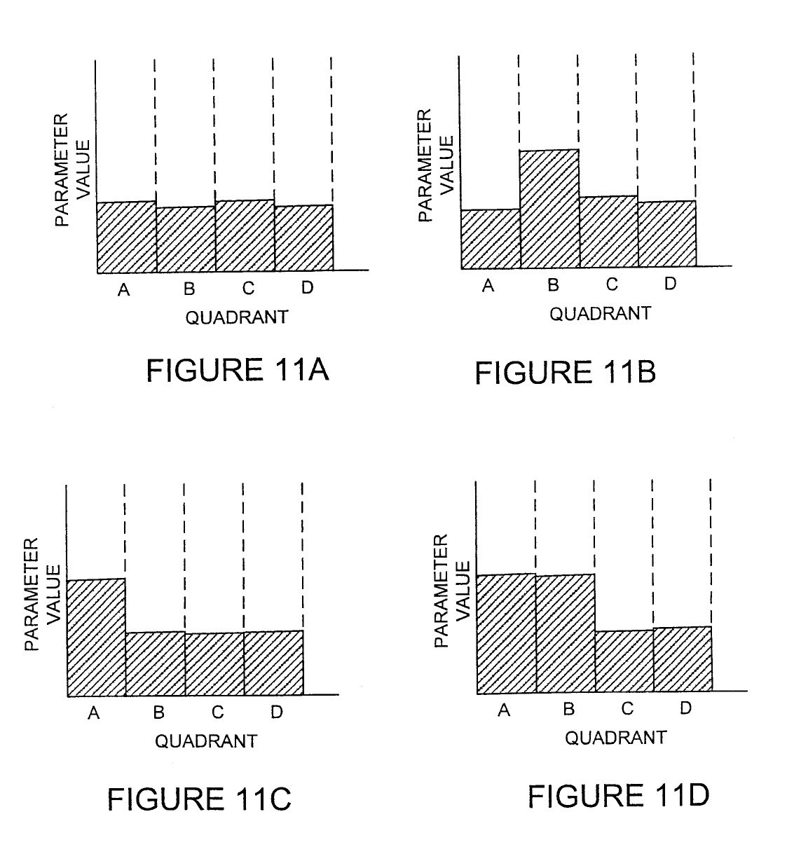

FIGS. 11A through 11D show schematically how parameters measured for different prostate quadrants by NIRS may vary. In FIG. 11A, a parameter value does not vary widely among the quadrants. FIGS. 11B and 11C each illustrate a situation where one quadrant exhibits a parameter value significantly higher than other quadrants. Where the parameter is indicative of blood flow or blood volume, the increase in the parameter for a quadrant may indicate increased vascularization in that quadrant. FIG. 11D shows a case where two quadrants show a higher parameter value and two other quadrants show a lower parameter value.

In some embodiments, controller 16 is configured to determine a parameter value for a plurality of different regions within a subject's tissues and to determine a degree of variation in the resulting set of parameter values. Controller 16 may store and/or display a graph or a value indicative of the degree of variation, non-uniformity or the like of values in the set of parameter values.

Where a component (e.g. a sensor, cable, sleeve, software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. For example:

Instead of a sleeve 12, other support means may be provided for supporting one or more sensors on a user's finger or an anal probe. For example, sensors 14 may be disposed on a self-adhesive patch that can be stuck onto the user's finger; sensors 14 may be held in place by one or more elastic rings or bands; sensors 14 may be disposed on one or more fingers of a glove; or the like.

apparatus as described herein may be applied to trans-vaginal probing. Catheter 40 may be a vaginal catheter in such an application.

1. An apparatus for evaluating one or more physiological conditions of a subject's tissue, the apparatus comprising:
one or more sensors;
support means for supporting the one or more sensors removably in place over a member insertable in a body cavity of the subject; and
a control unit operatively connected to the one or more sensors;
whereby when a user places the support means supporting the one or more sensors over the member in the body cavity in proximity to the subject's tissue, the one or more sensors can detect and transmit to the control unit signals relevant to the physiological condition of the subject's tissue.

2. An apparatus according to claim 1, wherein the support means comprise a sleeve.

3. An apparatus according to claim 2, wherein the sleeve is flexible.

4. An apparatus according to claim 3 wherein the sleeve comprises an elastic tube.

5. An apparatus according to claim 4 wherein a proximal end of the sleeve comprises one or more cords for facilitating placement of the support means over the member.

6. An apparatus according to claim 1 wherein the control unit is configured to process the signals to generate an image.

7. An apparatus according to claim 6 comprising a display operatively connected to the control unit to display the image.

8. An apparatus according to claim 2, wherein the member comprises a finger of the user and the sleeve is dimensioned to fit over the user's finger.

9. An apparatus according to claim 8, wherein the one or more sensors correspond in location on the sleeve to a ventral surface of the user's finger when the sleeve is worn by the user.

10. An apparatus according to claim 8, wherein the sleeve comprises an aperture corresponding in location on the sleeve to a sensitive part of the user's finger when the sleeve is worn by the user.

11. An apparatus according to claim 8, further comprising blocking means for preventing the one or more sensors from detecting signals relating to tissues in the user's finger.

12. An apparatus according to claim 11 wherein the blocking means comprises an opaque layer under the one or more sensors.

13. An apparatus according to claim 8 wherein the one or more sensors are directed away from the user's finger to avoid detection of signals relating to tissues in the user's finger.

14. An apparatus according to claim 2, wherein the member comprises an anal ultrasound probe and the sleeve is dimensioned to fit over the anal ultrasound probe.

15. An apparatus according to claim 14 wherein the sleeve is substantially acoustically transparent.

16. An apparatus according to claim 14 wherein the sleeve comprises one or more indicia alignable with one or more features on the anal ultrasound probe to facilitate repeatable positioning of the one or more sensors relative to ultrasonic transducers of the anal ultrasound probe.

17. An apparatus according to claim 6 wherein the image is superimposable on an ultrasound image generated by the anal ultrasound probe.

18. (canceled)

19. An apparatus according to claim 17 wherein the image comprises indicia representing one or more measurements derived from the signals by the control unit, wherein the one or more measurements relate to a parameter of the physiological condition of the subject's tissue, and wherein the indicia correspond to one or more locations in an image plane of the ultrasound image.

20. An apparatus according to claim 19 wherein the one or more locations comprise a line corresponding to locations in the subject's tissue where the one or more sensors are sensitive, and wherein the indicia comprises a graph indicating variation in the parameter as a function of position along the line.

21. An apparatus according to claim 1 wherein the one or more sensors are arranged in a two-dimensional array.

22. An apparatus according to claim 1 wherein the one or more sensors are configured such that the signals detected are indicative of the physiological conditions of the subject's tissue at a plurality of different distances from the one or more sensors.

23. An apparatus according to claim 1 wherein the one or more sensors are selected from the group consisting of: near infrared (NIR) light sensors, visible light sensors, ultrasound sensors, temperature sensors, pressure sensors, and combinations of two or more thereof.

24. An apparatus according to any of claims claim 6 wherein the one or more sensors comprise NIR light sensors.

25. (canceled)

26. An apparatus according to claim 24 wherein the NIR light sensors comprise optical fibres for carrying light detected at the sleeve to one or more light sensors of the control unit.

27. (canceled)

28. An apparatus according to claim 24 wherein the control unit is configured to determine from the signals one or more values indicative of concentrations of one or more biocompounds in the subject's tissue.

29. An apparatus according to claim 28 wherein the one or more biocompounds comprise biocompounds selected from the group consisting of: haemoglobin, oxygenated haemoglobin, non-oxygenated haemoglobin, cytochrome C oxydase and myoglobin.

30-33. (canceled)

34. An apparatus according to claim 24 wherein the control unit is configured to determine from the signals one or more values indicative of blood flow in at least one location in the subject's tissue.

35. An apparatus according to claim 34 wherein the image is indicative of the blood flow in the at least one location in the subject's tissue.

36. An apparatus according to claim 24 wherein the control unit is configured to determine from the signals one or more values indicative of blood flow at a plurality of locations in the subject's tissue.

37. (canceled)

38. An apparatus according to claim 24 wherein the NIR light sensors are arranged to have a spacing between adjacent sensors of about 1½ mm or less.

39-40. (canceled)

41. An apparatus according to claim 24 further comprising a plurality of NIR light sources operatively connected to the control unit.

42. An apparatus according to claim 41 wherein the NIR light sources emit radiation in multiple wavelength ranges.

43. An apparatus according to claim 41 wherein the NIR light sources are supported by the sleeve.

44. An apparatus according to claim 41 wherein the NIR light sources comprise optical fibres for carrying light from the control unit to the sleeve.

45. An apparatus according to claim 41 wherein each NIR light source is associated with an NIR light sensor and the apparatus comprises pairs of NIR light sources and associated NIR light sensors that are oriented in a plurality of directions.

46. (canceled)

47. An apparatus according to claim 41 wherein each NIR light source is associated with a plurality of NIR light sensors and the NIR light sources are spaced apart from associated NIR light sensors by a plurality of different distances.

48. (canceled)

49. An apparatus according to claim 41 comprising an element independent of the sleeve for supporting the NIR light sources.

50. (canceled)

51. An apparatus according to claim 49 wherein the element comprises a catheter.

52. An apparatus according to claim 41 wherein the NIR light sources comprise two or more independently controllable sets of NIR light sources.

53. An apparatus according to claim 1 wherein the signals are detected from different regions within the subject's tissue and wherein the control unit is configured to determine a parameter value for the signals.

54. An apparatus according to claim 53 wherein the different locations comprise quadrants of the subject's tissue.

55. An apparatus according to claim 53 wherein the control unit is configured to generate a value or plot indicative of the non-uniformity of the parameter values.

56. An apparatus according to claim 55 wherein the control unit is configured to store and/or display the value or the plot.

57. An apparatus according to claim 1 comprising a sheath for wearing over the holding member, the sheath constructed of a material that is substantially transparent to a form of energy detectable by the one or more sensors.

58. An apparatus for evaluating one or more physiological conditions of a subject's prostate and/or associated tissue, the apparatus comprising:
a sleeve dimensioned to be worn on a finger of a user, the sleeve comprising a proximal end open to receive the user's finger;
one or more sensors supported by the sleeve; and
a control unit operatively connectable to the one or more sensors;
whereby when the user places the one or more sensors supported by the sleeve on the user's finger in proximity to the subject's prostate, the one or more sensors can sense and transmit to the control unit information relevant to the one or more physiological conditions of the subject's prostate and/or associated tissues.

59. An apparatus according to claim 58 wherein the sleeve is flexible.

60. An apparatus according to claim 59 wherein the sleeve comprises a tube comprising an elastic material.

61. An apparatus according to claim 58, wherein the sleeve comprises an aperture corresponding in location on the sleeve to a sensitive part of the user's finger when the sleeve is worn by the user.

62. An apparatus according to claim 58, further comprising blocking means for preventing the one or more sensors from detecting signals relating to tissues in the user's finger.

63. An apparatus according to claim 58 wherein the one or more sensors are arranged in a two-dimensional array on the sleeve.

64. An apparatus according to claim 58, wherein the one or more sensors correspond in location on the sleeve to a ventral surface of the user's finger when the sleeve is worn by the user.

65. An apparatus according to claim 58 wherein the one or more sensors are selected from the group consisting of near infrared (NIR) light sensors, visible light sensors, ultrasound sensors, temperature sensors, pressure sensors, and any combination thereof.

66. An apparatus according to claim 58 wherein the one or more sensors comprise NIR light sensors.

67. An apparatus according to claim 66 further comprising a plurality of NIR light sources operatively connected to the control unit.

68. An apparatus according to claim 67 wherein the NIR light sources are supported by the sleeve.

69. An apparatus according to claim 67 comprising a urethral catheter wherein the NIR light sources are supported on the urethral catheter.

70. An apparatus for evaluating one or more physiological conditions of a subject's prostate and/or associated tissue, the apparatus comprising:
a sleeve dimensioned to be fitted over an anal ultrasound probe, the sleeve comprising a proximal end open to receive the anal ultrasound probe;
one or more sensors supported by the sleeve; and
a control unit operatively connected to the one or more sensors;
whereby when a user places the one or more sensors supported by the sleeve fitted over the anal ultrasound probe in proximity to the subject's prostate, the one or more sensors can sense and transmit to the control unit information relevant to the one or more physiological conditions of the subject's prostate and/or associated tissue.

71. An apparatus according to claim 70 wherein the sleeve comprises a material that is substantially acoustically transparent.

72. An apparatus according to claim 70 wherein the sleeve comprises one or more indicia alignable with one or more features on the anal ultrasound probe, whereby the location of the one or more sensors relative to ultrasonic transducers of the anal ultrasound probe is determinable.

73. An apparatus according to claim 70 wherein the one or more sensors are arranged in a two-dimensional array.

74. An apparatus according to claim 70 wherein the one or more sensors are selected from the group consisting of near infrared (NIR) light sensors, visible light sensors, temperature sensors, pressure sensors, and any combination thereof.

75. An apparatus according to claim 70 wherein the one or more sensors are NIR light sensors.

76. An apparatus according to claim 75 further comprising a plurality of NIR light sources operatively connected to the control unit.

77. An apparatus according to claim 76 wherein the NIR light sources are supported by the sleeve.

78. An apparatus according to claim 76 comprising a urethral catheter supporting the NIR light sources.

79. An apparatus for evaluating one or more physiological conditions of a subject's prostate and/or associated tissue, the apparatus comprising:
an anal probe,
a plurality of ultrasonic transducers supported by the anal probe;
a plurality of near infrared (NIR) light sensors supported by the anal probe;
an ultrasound control unit operatively connected to the ultrasonic transducers; and
an NIR light sensor control unit operatively connected to the NIR light sensors;
whereby when the probe is in proximity to the subject's prostate, the ultrasonic transducers are operable to obtain and transmit to the ultrasound control unit information for generating an ultrasound image of the subject's prostate and/or associated tissue, and the NIR light sensors are operable to sense and transmit to the NIR light sensor control unit information relevant to the one or more physiological conditions of the subject's prostate and/or associated tissue corresponding to the ultrasound image.

80. An apparatus according to claim 79 comprising a plurality of NIR light sources supported by the anal probe.

81. An apparatus according to claim 80 wherein the NIR light sensors and the NIR light sources are arranged in rows between blocks of the ultrasonic transducers.

82. An apparatus according to claim 80 wherein the NIR light sensors and the NIR light sources are interspersed among the ultrasonic transducers.

83. An apparatus according to claim 80 wherein the NIR light sensors and the NIR light sources are acoustically transparent and at least some of the NIR light sensors and the NIR light sources overlie the ultrasonic transducers.

84. (canceled)

85. An apparatus according to claim 80
wherein the ultrasonic transducers are substantially transparent at wavelengths detected by the NIR light sensors and at least some of the ultrasonic transducers overlie at least some of the NIR light sensors and the NIR light sources.

86. (canceled)

87. An apparatus according to claim 80 wherein each NIR light source is associated with an NIR light sensor.

88. An apparatus according to claim 87 wherein each pair of NIR light source and associated NIR light sensor are oriented in a direction different from another pair of NIR light source and associated NIR light sensor.

89. An apparatus for evaluating one or more physiological conditions of a subject's prostate and/or associated tissue, the apparatus comprising:
one or more near infrared (NIR) light sources positionable in the subject's urethra in or near the prostate;
one or more NIR sensors positionable in the subject's rectum; and
a control unit operatively connected to the one or more NIR light source and the one or more NIR sensors;
whereby the one or more NIR sensors can sense and transmit to the control unit information relevant to the one or more physiological conditions of the subject's prostate and/or associated tissues.

90. An anal probe for evaluating one or more physiological conditions of a subject's prostate and/or associated tissue comprising:
one or more near infrared (NIR) light sources;
one or more NIR sensors; and
a control unit operatively connected to the one or more NIR light source and the one or more NIR sensors;
whereby the one or more NIR sensors can sense and transmit to the control unit information relevant to the one or more physiological conditions of the subject's prostate and/or associated tissues.

91. An anal probe according to claim 90 further comprising ultrasonic transducers.

92. An anal probe according to claim 91 wherein the one or more NIR light source and the one or more NIR sensors are arranged in rows between blocks of the ultrasonic transducers.

93. An anal probe according to claim 91 wherein the one or more NIR light source and the one or more NIR sensors are interspersed among the ultrasound transducers.

94. An anal probe according to claim 91 wherein the one or more NIR light source and the one or more NIR sensors are acoustically transparent and overlie one or more of the ultrasound transducers.

95. An anal probe according to claim 91 wherein the ultrasound transducers are substantially transparent at wavelengths detected by the one or more NIR sensors.

96. An anal probe according to claim 90 wherein the one or more NIR light source and the one or more NIR sensors are arranged in bands that partially or entirely encircle the anal probe.

97. An anal probe according to claim 90 wherein the one or more NIR light source and the one or more NIR sensors are spirally arranged around the anal probe.

98. An anal probe according to claim 90 wherein the one or more NIR light source and the one or more NIR sensors are arranged in pairs wherein the pairs face in a plurality of different directions.

99-139. (canceled)
